(19) United States
(12) Patent Application Publication
(10) Pub. No.: US 2019/0240379 A1
(43) Pub. Date: Aug. 8, 2019

(54) NOVEL COMPOSITIONS ACTIVE ON ADIPOCYTES

(71) Applicant: BIOXIS PHARMACEUTICALS, Lyon (FR)

(72) Inventors: Frédéric BERTAINA, Poulx (FR); Alexandre GUERRY, Les Abrets (FR)

(73) Assignee: BIOXIS PHARMACEUTICALS, Lyon (FR)

(21) Appl. No.: 16/343,473

(22) PCT Filed: Oct. 24, 2017
(86) PCT No.: PCT/FR2017/052933

§ 371 Date: Apr. 19, 2019

(30) Foreign Application Priority Data
Oct. 25, 2016 (FR) 16 60346

(51) Int. Cl. A61L 27/60; A61L 27/20; A61L 27/54; A61K 45/06; A61P 17/00; A61K 9/00

(52) U.S. Cl. CPC A61L 27/60; A61L 27/20; A61L 27/54; A61K 31/722; A61P 17/00; A61K 9/0021; A61K 45/06

(57) ABSTRACT

An aqueous composition of chitosan including between 0.1 and 5 wt.-% chitosan relative to the total weight of the composition, for use in increasing the volume and/or number of adipocytes. The increase in the volume and/or number of adipocytes allows for use in the treatment of or combating against ageing of the skin, for filling in skin defeats, or the repair or reconstruction of continuous or subcutaneous tissues.

The present invention relates to the technical field of cosmetic fillers, biomaterials, physiological reconstruction materials for the skin, regenerative medicine, and reparatory surgery in particular for skin anti-ageing, in humans and optionally animals. In particular, the present invention relates to an aqueous chitosan composition aimed specifically at adipocytes. Another subject matter of the invention is such compositions for use thereof as a dermatological or cosmetic composition or as a medical device, advantageously as a bioresorbable implant.

Various filler products that can be injected in particular in humans are already known.

Collagen has for a long time been the product of choice as a filler product for the face, in particular for filling in lines and wrinkles or for redefining the lips. However, since hyaluronic acids were marketed, the latter are the most used. This is because the problems of safety relating to the animal origin (bovine or porcine) of collagen, there is added the biodegradability thereof that is judged to be too rapid. The direct injection of hyaluronic acid has the advantage of having an effect of immediate filling associated with a minor inflammatory reaction, because of its biocompatibility. However, the implantation thereof is associated with rapid biodegradation that makes the product unsatisfactory, even though the life of the injected product has been able to be extended by virtue of the use of crosslinked hyaluronic acids. Nevertheless, the products most used at the present time in aesthetic medicine and surgery are resorbable products the life of which is generally less than 12 months. Further products referred to as “permanent”, in that the bioresorption thereof may require several years, are also found on the market. These products contain among other things synthetic or biosynthetic polymers, such as acrylic derivatives and polyacrylamides, which give rise to significant fibrous encapsulation that is the cause of the longevity of the filling. However, the persistence of the products injected into the tissues presents a risk of complications in the long term or delayed-action inflammatory phenomena, for example the formation of inflammatory granulomas, cysts, etc., several months, or even several years, after the injection thereof.

Other synthetic products constitute an advantageous alternative: this is PLA (polylactic acid), a polymer the biodegradation of which is slower than that of other natural polymers, such as collagen or hyaluronic acid. It is considered in fact that the filling persists for up to two years after injection. These products have been marketed in particular under the name New-Fill® (or Sculptra®). The main problem with this technology is that the filling effect is visible only after a period of eight weeks, which does not provide complete satisfaction for the patient. Moreover, the fibrosis observed in the use of non-degradable products has appeared of great interest in terms of long-term aesthetic effect, and thus filler products referred to as “semi-permanent” have been developed which, through their heterogeneous “particle-vector” composition, have a pro-fibrotic effect while remaining biodegradable. Mention can be made for example of the product Atlean®, which offers a dispersion of TCP (tricalcium phosphate) particles in hyaluronic acid, and the product Radiesse®, which offers a dispersion of calcium hydroxyapatite particles in a carboxymethyl cellulose gel. In all cases, the vector gel provides the aesthetic effect of immediate filling, while the particles little by little cause fibrosis that guarantees the long-term effect. The advantage of these products, apart from this double action mechanism (mechanical and tissue inducing) is that they are in the end completely resorbed.

In this context, chitosan is another molecule that has been of interest in the field. This is because chitosan, because of its unique biomimetic chemical structure, behaves vis-à-vis the organism as a “decoy” for the biological environment (A. Montembault, K. Tahiri, C. Korwin-Zmijowska, X, Chevalier, M. Corvol, A. Domard, Biochimie, 88 (2006), 551-64): on the one hand it is sufficiently “recognised” for not causing a dangerous inflammatory reaction, and on the other hand sufficiently “unrecognised” not to be degraded too quickly.

The molecule in fact consists of a succession of N-acetyl-D-glucosamine and D-glucosamine units, the first being a constituent of molecules of the extracellular matrix (this residue is found in hyaluronic acid for example), and the second being completely absent therefrom, and chitosan is therefore more difficult to degrade from a biological point of view. Moreover, chitosan is known in the literature for stimulating some immune cells, such as macrophages, which produce in its presence an increased quantity of growth factors. These growth factors are biological mediators that promote the production of extracellular matrix and the proliferation of fibroblasts, cells producing collagen fibres. Thus chitosan promotes the synthesis of fibrous tissue, which affords a “biological” filling in the long term, in particular filling of skin defects and cavities in the human body or face, such as wrinkles. The applicant previously closely followed chitosan with a view to the aforementioned application, which was the subject of a patent application WO 2013/07964.

Nevertheless, despite these advances, there still exists a need for molecules and means for treating the skin, affording the filling and treatment of wrinkles and lines and more generally having an anti-ageing action and/or physiological skin reconstruction in humans and optionally animals. In addition, equivalent compositions capable of treating larger skin depressions, following surgery or an illness, are required by practitioners.

Thus, completely surprisingly, the applicant realised that an unexpected additional technical effect on the skin appeared when a specific aqueous composition of chitosan, unknown until the present time, was used. This is because a more meticulous study revealed that, when a chitosan solution at a particular concentration of this compound was used, there was a direct action on the adipocytes present in the skin layers. Thus the adipocytes subjected to this treatment proliferate and swell in a controlled manner, and enable the skin to firm by filling in wrinkles and lines or provide the filling of skin defects or the volumetric reconstruction of tissues. This finding led the applicant to take an interest particularly in adipocytes. Thus several points were revealed by the applicant. He perceived first of all that an excessively high concentration of chitosan, without removing its other benefits, could nevertheless prove toxic for adipocytes. It was also observed by the applicant that the presence of at least one component of the extracellular matrix in the aqueous composition makes it possible to increase the capture of fatty acid of the adipocytes. In vivo, the chitosan composition according to the present invention induces the synthesis of collagen fibres. In the light of the information collected by the applicant, the addition of at least one component of the extracellular matrix (or a substituent) makes it possible to potentiate the action of chitosan.

Thus the applicant proposes aqueous compositions having a direction action on the adipocytes at a (sub)cutaneous level making it possible to add an original technical solution to the art in the field of skin treatment and reconstructive surgery.

### SUMMARY OF THE INVENTION

The subject matter of the present invention relates to an aqueous chitosan composition comprising between 0.1% and 5% by weight chitosan with respect to the total weight of the composition, for use thereof in increasing the volume and/or number of adipocytes.

In particular, the increase in the volume and/or number of adipocytes thus obtained allows use in the treatment of or combating against ageing of the skin, for filling in skin defects such as wrinkles or the repair or reconstruction of cutaneous or subcutaneous tissues.

In other words, the subject matter of the present invention is the use of an aqueous chitosan composition comprising between 0.1% and 5% by weight chitosan with respect to the total weight of the composition, in order to increase the volume and/or number of adipocytes. In particular, this use is preferably cosmetic and non-therapeutic.

This is because, apart from the fact that an injection or the introduction of an implant according to the present invention will in fact fill in a volume at the cutaneous or subcutaneous level through the inherent volume of the composition, said composition according to the present invention will furthermore cause a local swelling by virtue of the action thereof on the adipocytes (increasing the number and/or the volume) around this composition. Thus the aqueous composition according to the present invention can be used for filling in cavities in the body or face, such as wrinkles or lines, for the creation or the increase in volumes of the face or human body, or for healing the skin.

Adipocytes are commonly known for being animal cells present in the adipose tissues, and specialising in the storage of fat. Furthermore, adipocytes are also included in the visceral tissue and in the hypodermis, which is the deep layer of the skin, containing more or less adipose tissue, serving as an interface between the dermis and the mobile structure situated below it (organs, muscles, etc.). Thus the subcutaneous adipocytes are situated at a variable depth from the surface of the skin, varying from a few millimetres to several centimetres in depth. Preferably, “adipocytes” in the context of the present invention means human adipocytes.

The subject matter of the present invention further relates to an aqueous composition as described at present, for use thereof as a dermatological composition or as a medical device, advantageously as a bioresorbable or partially bioresorbable implant for increasing the volume and/or number of adipocytes.

“Bioresorbable” or “bioresorption” means a biodegradation that results in the total or mainly total degradation of the product injected. Advantageously, according to the present invention, the aqueous composition has good biocompatibility and is bioresorbable. In particular, the product according to the invention has a longer duration of bioresorption than products based on hyaluronic acid of the crosslinked hyaluronic acid type, for a prolonged effect, such as a prolonged filling effect.

Another subject matter of the present invention is the use of at least one component of the extracellular matrix, and/or at least one substituent of the extracellular matrix, for reducing toxicity with regard to the adipocytes of an aqueous composition of chitosan comprising between 0.1% and 5% by weight chitosan with respect to the total weight of the composition.

### Composition

The subject matter of the present invention is an aqueous chitosan composition.

“Aqueous composition” means a composition comprising at least 1% by weight water, advantageously at least 50% by weight water, even more advantageously at least 80% by weight water.

The percentages, in general for the present invention, when they are not specified, correspond to percentages by weight with respect to the total weight of the composition in question.

Preferably, the aqueous composition of chitosan according to the invention has a pH of less than 8.5, advantageously between 4 and 7.6. However, when it is wished to inject the chitosan composition directly into the tissues that are at the physiological pH (between 6.8 and 7.4), it would be desirable to use a composition having a pH as close as possible to the physiological pH in order to avoid necrosis of the tissues because of the acidity of the formulation injected. In a particular embodiment according to the present invention, the pH of said solution is greater than or equal to 5.5, advantageously between 5.5 and 7.5, preferably between 6.2 and 6.8.

The aqueous composition according to the present invention is advantageously an aqueous chitosan solution, a gelling aqueous chitosan solution or an aqueous chitosan gel.

“Solution” means, within the meaning of the present invention, a composition in liquid form, in contradistinction to a gelled composition. A solution has a liquid phase containing at least two chemical species. An “aqueous solution” comprises at least 1% by weight water, advantageously at least 50% by weight water, even more advantageously at least 80% by weight water. Preferably, an aqueous solution according to the present invention comprises mainly water, with optionally other compounds. Advantageously, the aqueous solution according to the present invention is an aqueous chitosan solution.

Advantageously, the aqueous solution according to the present invention is a homogeneous aqueous chitosan solution. “Homogeneous aqueous chitosan solution” means, within the meaning of the present invention, that all the chitosan polymer is solubilised and that the solution does not contain any solid chitosan particles in suspension in the liquid phase. The aqueous solution thus formed comprises at least water in the proportions mentioned above and chitosan with optionally other compounds. The homogeneous aqueous chitosan solution according to the present invention is typically transparent.

The aqueous chitosan composition according to the present invention can be used to form a gel.

As for “gel”, it can be accepted according to the present invention that it is a body having a storage modulus greater than the loss modulus (cf. H. H. Winter, F. Chambon, Analysis of linear viscoelasticity of a crosslinking polymer at the gel point, J. Rheol., 30 (1986), pp. 367-382). The stability and integrity of this body are maintained either by non-covalent interactions (physical gel) or by a covalent chemical crosslinking (chemical gel).

This gel can be formed by any technique known in the art, for example by a variation in pH or temperature. In a particular embodiment, the aqueous composition according to the present invention is gelled at a pH of between 6 and 9, advantageously between 6.5 and 8.5, more advantageously between 7 and 8.

Preferably, the aqueous chitosan solution (whether it be homogeneous or not) is first of all prepared and then gelled.

“Gelling” aqueous chitosan composition or solution means, in the context of the present invention, an aqueous chitosan composition or solution as defined above, able to provide an aqueous chitosan gel, in particular when it is injected into the biological medium.

Thus one subject matter according to the present invention relates to a gel obtained from the aqueous composition or solution as described above. In a particular embodiment of the present invention, this gel is formed before incorporation thereof in a composition as described herein.

In another particular embodiment according to the present invention, this gel is formed after incorporation thereof in a composition. This can be carried out by varying the pH (change to a physiological pH, i.e. between 6.8 and 7.8, preferentially at a pH of 7.4±0.2).

Particularly advantageously, the aqueous composition according to the invention can be injected into the human or animal body, typically by intradermal, subcutaneous or intraperitoneal injection. The composition may be packaged in a syringe, such as a sterile syringe.

Thus the aqueous composition has a suitable viscosity for providing good syringeability (satisfactory flow through a needle in a syringe) and ease of injection. In a particular embodiment, the aqueous composition according to the invention is sterilised before injection, for example by autoclaving.

Thus the composition according to the invention can be formulated so as to be administered by intradermal injection or by subcutaneous or intraperitoneal injection.

Intradermal injection is the administration of a compound in the dermis of the skin, situated just below the epidermis. Subcutaneous injection consists of introducing a needle into the subcutaneous layer of the skin, in order to inject therein a molecule of interest. Intraperitoneal injection is located beyond the peritoneum in the abdominal cavity, i.e. in the adipovisceral tissue.

In a particular embodiment, the aqueous composition according to the present invention is included in or consists of an implant for treating skin depressions. It may be a case of skin depressions of small volumes (i.e. equal to or less than 5 ml approximately equivalent to 5 grams according to the density of the composition) or which cannot be filled in by small-volume injections. This is the case for example with filling the ablation of an organ or a tissue following an accident or an illness (e.g. mammoplasty, acute lipodystrophy, acute lipoatrophy, filling treatment following a poliomyelitis), in reparatory surgery.

According to a particular feature of the present invention, the chitosan composition has a long resorption time once injected, typically from a few weeks to several months, for example around 3 to 4 weeks up to 12 to 18 months. The product or biomaterial consisting of or containing the aqueous composition according to the invention benefits from the bacterio- and fungistatic character of chitosan, well known in the world of the food industry and healing dressings. These properties facilitate the storage of the product and help to limit the risks of infection related to the injection or the long-term inflammatory phenomena for other products as mentioned above. Compared with the natural molecules (collagen, hyaluronic acid) used at the present time for filling in depressions or volume reconstruction (e.g. wrinkles, lines, mammoplasty or lipodystrophy), chitosan is the only one to have such properties. Moreover, the product or biomaterial consisting of or containing the aqueous composition according to the invention provides effective biological filling that is advantageously immediate. Furthermore, chitosan can promote the synthesis of collagen and provide filling of skin defects, such as wrinkles, by stimulating natural mechanisms.

Furthermore, the aqueous composition according to the present invention is advantageously able to form crystalline chitosan particles after injection. This is because in this way the effects as described previously in WO 2013/07964 are cumulative with the effects relating to the present invention.

### Chitosan

The composition according to the invention comprises at least one chitosan.

Chitosan is an amino-polysaccharide generally obtained by N-deacetylation of chitin, a polysaccharide that is as widespread in the biomass as cellulose. Chitin is in particular present in the cuticles of arthropods, the endoskeleton of cephalopods and the cell walls of fungi, yeasts or algae. Advantageously, according to the present invention, chitosan is a natural product that comes from an animal source, for example crustaceans of the crab, prawn or squid type, or from a vegetable source, such as fungi or algae. Chitosan and chitin are linear copolymers respectively of 2-acetamido-2-deoxy-D-glucan and 2-amino-2-deoxy-D-glucan. N-acetyl-D-glucosamine (GlcNAc) and D-glucosamine (GlcN) units are more commonly spoken of, bonded by glycosidic β(1→4) bonds. Chitin and chitosan are differentiated through the molar fraction (expressed as %) of the GlcNAc units present in the copolymer, also referred to as the degree of acetylation (DA).

The chemical structures of chitosan and chitin are shown schematically below according to the degree of acetylation (DA):

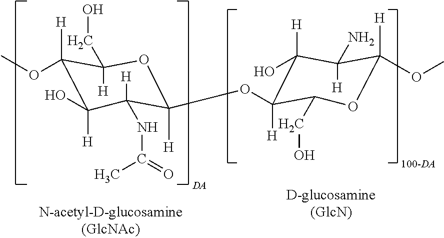

The DA is calculated by means of the formula:

$$D A ( \% ) = \frac{nGlcNAc}{nGlcNAc + nGlcN} \times 100$$

with nGlcNAc=number of acetylated units and nGlcN=number of deacetylated units.

The DA must thus be determined according to the standards currently in force (namely “Standard Guide for Characterization and Testing of Chitosan Salts as Starting Materials Intended for Use in Biomedical and Tissue-Engineered Medical Product Applications, Book of Standards Volume: volume 13.01; ASTM-F2103”).

Advantageously, according to the present invention, chitosan has a degree of acetylation (DA) of less than 30%, even more advantageously less than or equal to 20%, for example less than 15%. Preferably, the chitosan according to the invention has a degree of acetylation DA of less than or equal to 15%, advantageously less than 10%, more advantageously less than 8%, even more advantageously less than 5%. Even more preferably, the chitosan according to the invention has a degree of acetylation DA of less than or equal to 4%, advantageously less than 3%, more advantageously less than 2%, even more advantageously less than 1%. For example, the chitosan according to the invention has a degree of acetylation DA of between 1% and 20%, advantageously between 2% and 15%, advantageously between 3% and 10%, for example between 4% and 8%.

The degree of acetylation is preferably measured according to the method described in the publication “Physico-chemical studies of the gelation of chitosan in a hydroalcoholic medium” A. Montembault, C. Viton, A. Domard, Biomaterials, 26(8), 933-943, 2005).

The chitosan used in the solutions of the invention preferably has a weight-average molecular weight (determined before sterilisation according to the method described in “Physico-chemical studies of the gelation of chitosan in a hydroalcoholic medium” A. Montembault, C. Viton, A. Domard, Biomaterials, 26(8), 933-943, 2005) of between 100,000 and 1,500,000 g/mol, advantageously between 200,000 and 1,000,000 g/mol, more advantageously between 250,000 and 500,000 g/mol, and even more advantageously between 300,000 and 400,000 g/mol.

After sterilisation, the chitosan typically has a weight-average molecular weight of between 80,000 and 1,000,000 g/mol, and advantageously between 120,000 and 300,000 g/mol.

Conventionally a chitosan with a molar mass of between 100,000 and 1,000,000 g·mol⁻¹ can also be characterised by its viscosity (conventionally at a concentration of 1% in a 1% aqueous solution in acetic acid at 25° C.). With this consideration, the molar mass of chitosan can also be defined by a viscosity of between 5 Pa·s and 20 Pa·s.

The viscosity of the composition is measured at 25° C. by means of a DHRI rheometer (TA Industries) and a planar geometry of 40 mm in accordance with a dynamic mode with shear rate applied of 0.01 to 1 s⁻¹.

According to a preferred embodiment, the chitosan used in the solutions according to the invention is not modified chemically, and in particular is not grafted in order to promote its solubility in an aqueous solution at pHs of close to neutrality (between 6.2 and 7.2). It is distinguished in this regard from the chitosans used in the application WO 03/042250 or the application JP-H02-69502, in the publication “Synthesis and characterization of sugar-bearing chitosan derivatives: aqueous solubility and biodegradability”, Jae Hyung Park et al., Biomacromolecules 2003, 4, 1087-1091, and in the publication “Water solubility of partially N-acetylated chitosans as a function of pH: effect of chemical composition and depolymerization” Varum K. M. et al., Carbohydrate Polymers 25 (1994), 65-70.

In this embodiment, this other chitosan may be in the form of crosslinked chitosan particles.

According to a particular embodiment, the composition according to the invention is free from chitosan having a high degree of acetylation, in particular greater than 20% and more specifically between 40% and 60%.

According to another particular embodiment, the composition according to the invention is free from additional oligomer, in particular a chitosan oligomer, with a low mean molecular weight, in particular less than 20,000 g/mol.

“Free” from chitosan having a high degree of acetylation or from an additional oligomer means, within the meaning of the present invention, that the aqueous chitosan composition comprises less than 1% by weight of these compounds, in particular less than 0.5% by weight, and more preferentially does not contain these compounds.

### Dispersion Agent

Apart from the chitosan, the aqueous composition according to the present invention may comprise at least one chitosan dispersion agent. This type of dispersion agent is very well known in the art and can for example be chosen from mannitol, glycerol, sorbitol and mixtures thereof.

### Buffer

The composition according to the invention may also comprise a pH buffer in order to be placed at a physiological pH. Any pH buffer commonly used for this purpose can be used, for example PBS (phosphate buffer saline).

### Salt

In a particular embodiment, the composition according to the invention comprises a salt such as sodium chloride or potassium chloride, or any other excipient advantageously acceptable for adjusting the osmolarity of the composition. The addition of a salt such as sodium or potassium chloride may be advantageous for obtaining an isotonic solution. According to a particular feature of the present invention, the composition may also comprise at least one compound having a recognised therapeutic activity.

### Additional Compound or Excipient

The aqueous chitosan composition according to the present invention may also comprise at least one pharmaceutically, dermatologically and/or cosmetically acceptable compound or excipient.

### Active Agents

In addition the composition according to the present invention may comprise at least one active compound such as an analgesic compound, a local anaesthetic such as lidocaine, mepivacaine, bupivacaine or ropivacaine, an angiogenic compound, or an active compound of the growth factor or bioactive oligosaccharide type.

### Compound or Substituent of the Extracellular Matrix

According to a particular embodiment, the composition according to the invention may comprise a compound or substituent of the extracellular matrix.

“Extracellular matrix” designates typically all the extracellular macromolecules of the connective tissue and other tissues. One compound of the extracellular matrix according to the present invention is advantageously an organic polymer, for example with a size greater than 1000 daltons (Da), or greater than 5000 Da. Advantageously, the organic polymers of the extracellular matrix according to the present invention may be of a protein, glucoside, proteoglucoside or glucoprotein nature. Typically the organic polymers of the extracellular matrix may be collagen, hyaluronic acid or fibronectin. A “substituent of the extracellular matrix” is a non-ubiquitous compound (i.e. one that is not found naturally in humans for this function) making it possible to fulfil the role of extracellular matrix. This type of compound is known in the art (Jayakumar, R., Menon, D., Manzoor, K., Nair, S. V., & Tamura, H. (2010). Biomedical applications of chitin and chitosan based nanomaterials—A short review. Carbohydrate Polymers, 82(2), 227-232).

The compound of the extracellular matrix is preferably chosen from proteins, glycosaminoglycans and mixtures thereof.

The proteins and the glycosylated derivatives thereof can preferably be chosen from collagen, elastin, fibronectin, laminin or mixtures thereof.

The glycosaminoglycans can preferably be chosen from hyaluronic acid, chondroitin sulphate, heparan sulphate, keratan sulphate or a mixture of at least two of these glycosaminoglycans.

According to a preferred embodiment, the compound of the extracellular matrix is chosen from proteins and the glycosylated derivatives thereof, preferably from collagen, fibronectin and a mixture of collagen and fibronectin.

The substituent of the extracellular matrix for its part can be chosen from carboxymethyl cellulose, chitosan ester, chitin ester and a mixture of at least two substituents of the extracellular matrix.

According to a preferred embodiment, when the composition according to the invention comprises a compound or a substituent of the extracellular matrix, it is free from an additional oligomer, in particular a chitosan oligomer with a low mean molecular weight, in particular less than 20,000 g/mol.

The aqueous composition according to the present invention preferentially contains less than 5% by weight, with respect to the total weight of the composition, of at least one compound of the extracellular matrix and/or of at least one substituent of the extracellular matrix, in particular between 0.01% and 5% by weight, advantageously between 0.1% and 4% by weight, more advantageously between 0.2% and 3% by weight, even more advantageously between 0.3% and 2% by weight, or even between 0.5% and 1% by weight.

The homogeneous aqueous chitosan composition according to the present invention, when it comprises a compound of the extracellular matrix and/or a substituent of the extracellular matrix, preferably contains a quantity of chitosan less than or equal to 5% by weight with respect to the total weight of the aqueous composition, advantageously less than or equal to 4% by weight with respect to the total weight of the aqueous composition, less than or equal to 3% by weight with respect to the total weight of the aqueous composition, even less than or equal to 2% by weight with respect to the total weight of the aqueous composition. More preferably, the homogeneous aqueous chitosan composition according to the present invention, when it comprises a compound of the extracellular matrix and/or a substituent of the extracellular matrix, contains between 0.1% and 5%, advantageously between 0.5% and 3.5%, in particular between 1% and 2%, by weight of chitosan, with respect to the total weight of the aqueous composition. More preferably, the homogeneous aqueous chitosan composition according to the present invention, when it comprises a compound of the extracellular matrix and/or a substituent of the extracellular matrix, contains between 1.5% and 2%, advantageously between 1.6% and 1.9%, in particular between 1.7% and 1.8% chitosan by weight with respect to the total weight of the aqueous composition. Even more preferably, the homogeneous aqueous chitosan composition according to the present invention, when it comprises a compound of the extracellular matrix and/or a substituent of the extracellular matrix, contains less than 2%, advantageously less than 1.9%, in particular less than 1.8%, more advantageously less than 1.7%, even more advantageously less than 1.6% chitosan by weight with respect to the total weight of the aqueous composition.

### Method for Preparing the Compositions

The aqueous chitosan composition according to the invention, as described herein, can in particular be prepared by the following successive steps:

a. dissolution of chitosan in water by adding acid such as a weak acid, said weak acid advantageously being chosen from the group consisting of acetic acid, glyconic acid, lactic acid or a glutamic acid, or such as a strong acid, said strong acid advantageously being hydrochloric acid, and mixtures thereof,

b. the addition where applicable of at least one compound and/or substituent of the extracellular matrix, and

c. optional readjustment of the pH in order to obtain an aqueous composition having a pH of less than 8.5.

Preferably, at least one dispersion agent (such as mannitol, glycerol, sorbitol, etc.) is added where applicable at any of steps (a), (b) or (c) or after any of steps (a), (b) or (c) above. In a particular embodiment according to the present invention, the aqueous chitosan composition, gelled, may be dispersed by the addition of a dispersion agent as presented above, optionally with a step of mechanical fragmentation of the gel of said aqueous chitosan composition. The composition obtained may in all cases be a suspension or a solution, able to be administered for example by injection, preferentially gelling.

In a particular embodiment, the aqueous composition of the present invention is able to be obtained by the steps described above.

Controlling the pH of the solutions is very important in order firstly to avoid acid necrosis of the tissues after injection, as explained above, and also to protect the compositions from hydrolysis and degradation of the chitosan if sterilisation is used (for example by autoclave at 121° C. for 15 minutes). The pH is readjusted if necessary with a compound such as sodium bicarbonate or a PBS buffer, typically in small quantities. The pH is advantageously checked by means of a pH meter.

In the context of the present invention, the pH can preferably be adjusted very gradually by dialysis.

Dialysis is a method for the membrane separation of molecules and ions in solution. Thus, in the context of the present application, the aqueous chitosan solution according to the invention can be dialysed against a buffer solution having a pH equal to the final pH required for the chitosan solution (target pH), that is to say at least greater than 6.2, advantageously between 6.2 and 7.2, preferably between 6.25 and 7.1. When the buffer solution has a pH higher than the gelling pH of the solution (for example 7.5), the dialysis should then be monitored in order to prevent gelling of the solution.

The buffer solution may, for example, be a saline phosphate buffer solution (PBS, TBS, PBS-lactic acid), tris (tris(hydroxymethyl)methylamine), 4-2-hydroxyethyl-1-piperazineethanesulfonic acid (HEPES), 2-{[tris(hydroxymethyl)methyl]amino}-ethanesulfonic acid (TES), 3-(N-morpholino)propanesulfonic acid (MOPS), piperazine-N,N′-bis(2-ethanesulfonic acid)), MES (2-(N-morpholino)ethanesulfonic acid (PIPES), sodium chloride (NaCl).

According to a preferred embodiment, the buffer solution is a PBS (phosphate buffer saline) solution composed of an “acid” salt NaH₂PO₄, a “basic” salt Na₂HPO₄ and NaCl.

According to a particular embodiment, the buffer is physiologically acceptable, that is to say it does not present any risk of intolerance or toxicity when the injectable solution according to the invention is injected into the tissues. In this regard, the buffer is preferably different from glycerophosphate, in particular β-glycerophosphate, which, although not very irritant for the skin, poses problems of calcification when it is injected into tissues.

According to a particular embodiment of the invention, the dialysis can be carried out statically in a single bath against a buffer solution as described previously. When the dialysis is carried out statically in a single bath against a buffer solution, it is preferable for said buffer solution to have a pH equal to the final pH required for the chitosan solution (target pH), for example between 6.5 and 6.9.

According to a more preferred embodiment, the dialysis can be carried out statically in a plurality of successive baths against buffer solutions having different pHs and closer to the final pH required for the chitosan solution (target pH).

According to a particularly preferred embodiment, the dialysis is carried out dynamically, that is to say by continuously circulating at least one solution affording the gradual increase in the pH through one or more dialysis pockets consisting of a dialysis membrane containing the aqueous chitosan solution. This type of dialysis is for example described in the patent application WO 2016/170284 of the applicant.

In a particular embodiment according to the invention, the chitosan is dissolved in water using a strong acid of the hydrochloric acid type. In this case, the pH is readjusted with a compound of the sodium or ammonium bicarbonate type or PBS for example, and/or a base of the NaOH or KOH type for example (always controlling the pH so that it remains below 8.5, preferentially below 6.9).

In a particular embodiment according to the invention, during the dissolution step, the acid is added in a quantity necessary for the dissolution of the chitosan. In this way it is possible to use an excess of acid for some chitosans, for example chitosans that are difficult to solubilise with the strictly necessary quantity of acid, and then the chitosan is reprecipitated, using ammonia for example. After a series of washings intended to eliminate the excess ammonia and the salts, it is then possible to lyophilise the chitosan in order to recover the dry matter. This will then be easier to solubilise.

In another particular embodiment according to the invention, during the dissolution step, the acid is added in a quantity at least necessary for the dissolution of the chitosan, such as the stoichiometric quantity strictly necessary for the protonation of the NH₂ sites.

Particularly advantageously, the pH of the aqueous composition according to the present invention is below 8.5, and is typically between 4 and 7.6. In the context of the invention, the chitosan is solubilised in an aqueous solution, such as water, in an acid environment in the pH ranges mentioned previously, advantageously by protonation of the amine groups of the chitosan. These pH ranges have in particular been chosen in order to maximise the stability of the aqueous composition according to the invention.

The subject matter of the present invention also relates to an aqueous composition, as described above, characterised in that said composition is liquid, in gel form or obtained after dispersion of said aqueous chitosan composition in a dermatologically and/or cosmetically acceptable vehicle.

“Dermatologically and/or cosmetically acceptable vehicle” means, in the context of the present invention, any dermatologically and/or cosmetically acceptable excipient, or any dermatologically and/or cosmetically acceptable matrix or device, allowing administration of the aqueous composition according to the present invention.

When an aqueous composition as described herein is included in a composition with optionally a pharmaceutically, dermatologically and/or cosmetically acceptable compound or excipient, the use thereof as a dermatological composition or as a medical device, advantageously as a bioresorbable or partially bioresorbable implant, can be administered at the following doses. The quantity of chitosan may be between 0.1% and 5% by weight, advantageously between 1% and 2% by weight, more advantageously between 1.1% and 1.9% by weight, even more advantageously between 1.2% and 1.8% by weight, and even more advantageously between 1.3% and 1.7% by weight or between 1.4% and 1.6% by weight, for example at 1.5%±0.05% by weight chitosan with respect to the total quantity of aqueous composition administered. Furthermore, the quantity of composition or aqueous composition administered in a single dose is between 0.01 and 20 grams per injection, advantageously between 0.05 and 10 grams per injection, more advantageously between 0.1 and 5 grams per injection, even more advantageously between 0.4 and 1 gram per injection, for example 0.7 grams±0.1 grams per injection. The number of injections may vary from 1 to 20 times in the most extreme cases. More routinely, this number of injections is less than 5 times for effectively filling in cavities with a volume of less than 50 ml (and therefore at physiological temperature). In the case of reparatory surgery greater than mentioned previously (e.g. mammoplasty, acute lipodystrophy, acute lipoatrophy, etc.) the quantities of composition according to the present invention that can be injected and/or implanted can be added to masses equal to or less than 500 grams, more commonly equal to or less than 350 grams, even more commonly equal to or less than 200 grams, or equal to or less than 100 grams. Typically, the density of the aqueous composition administered is between 0.9 and 1.2 at 20° C. (and therefore before administration).

The cosmetic use according to the present invention is defined in fact as not being invasive in the administration of the composition according to the present invention. This excludes any surgical act. Nevertheless, the cosmetic use according to the present invention may be carried out with small-calibre needles and syringes, in an act comparable to the application of a tattoo. Thus, in an arbitrary fashion, injected quantities of composition according to the present invention greater than 5 grams per injection are excluded in the context of cosmetic use. In this context, the number of injections is limited arbitrarily to no more than 5 times for a total injected quantity of 10 grams.

Furthermore, the quantity of aqueous composition administered in a single dose may be between 0.001 and 1 gram per cm² of skin surface, advantageously between 0.01 and 0.5 grams per cm² of skin surface. Typically, the density of the aqueous composition administered is between 0.9 and 1.2 at 25° C.

Furthermore, the use according to the present invention can be carried out in vitro or in vivo.

In vitro use is applicable to all applications to which the present invention relates, and in particular for purposes of determining concentrations/quantity of active agents to be administered.

### DESCRIPTION OF THE FIGURES

FIGS. 1A and 1B: effect on the proliferation of 3T3L1s. FIG. 1 shows that, for all concentrations, the 3T3L1 cells are capable of proliferating and reaching confluence in 5 days although, two days after seeding, the morphology of the cells suggests an aggressive or even toxic medium for the 2% condition. FIG. 1B specifies where, on the experimental images obtained, the signal corresponding to AdipoRed could be seen (green experimentally, versus red for Hoecht).

FIGS. 2A, 2B and 2C: these figures show the influence of the chitosan concentration on the storage of oleic acid by the 3T3L1 cells during adipocyte differentiation. The AdipoRed marking (FIG. 2A) shows an accumulation of fatty acids when the cells are in the presence of chitosan at 0.5%, 0.75% and 1% compared with the conditions not containing chitosan. FIG. 2B specifies where, on the experimental images obtained, the signal corresponding to Adipored could be seen (green experimentally). Quantification of this accumulation (FIG. 2C) demonstrates that it is maximum when the cells are cultivated in the presence of chitosan at 0.5%.

FIG. 3: this figure shows a distribution of the lipid droplets (n>10000) of the 3T3L1s set out a composition of chitosan and at a formulation comprising chitosan combined with collagen and fibronectin. FIG. 3 shows that (Col 2+FN1)+Chitosan 2%) are the best formulations for increasing the number and average size (volume) of the lipid droplets.

### EXAMPLES

### Example 1: Preparation of the Solutions

Two strategies for preparing a range of concentrations of gelling chitosan solutions are used.

### By Final Dilution

4 g of chitosan is stirred in 95 g of saline solution containing 150 mM of NaCl (equivalent to 300 mOsmol·l⁻¹). Once the chitosan is completely dispersed, 1 g of acetic acid is added. Stirring is maintained until the chitosan is completely dissolved. A clear solution of 4% concentrated chitosan is then obtained with a pH that may lie between 3 and 4.5 according to the origin of the chitosan.

A 4% (w/w) chitosan solution is obtained in a saline solution (NaCl at 300 mOsmol·l⁻¹) containing 1% (w/w) of acetic acid.

The pH of the acidic chitosan solution is increased until it is between 6 and 6.5. This step is carried out by dialysis against a PBS buffer having a pH of between 6.5 and 7. During this step, the swelling of the chitosan solution is controlled in order to reach a final concentration of 3% (w/w) (degree of swelling 33%). The chitosan solutions are generally sterilised (121° C. for 15 minutes in an autoclave) in order to be stored before use.

This solution is dialysed in order to obtain a pH close to 6 and a concentration of 3% (w/w).

Before use, the 3% chitosan solution can be diluted in a saline solution at 150 mM of NaCl+1% acetic acid in order to access lower concentrations.

The solutions obtained can gel in the presence of the physiological medium or a culture medium.

### By Initial Dilution

4 g of chitosan is stirred in 95 g of saline solution containing 150 mM of NaCl (equivalent to 300 mOsmol·l⁻¹). Once the chitosan is completely dispersed, 1 g of acetic acid is added. Stirring is maintained until the chitosan is completely dissolved. A clear 4% concentrated chitosan solution is then obtained with a pH that can be between 3 and 4.5 according to the origin of the chitosan.

A 4% (w/w) chitosan solution is obtained in a saline solution (NaCl at 300 mOsmol·l⁻¹) containing 1% (w/w) of acetic acid.

This mother solution (4%) is next diluted in a saline solution (NaCl at 300 mOsmol·l⁻¹) containing 1% (w/w) of acetic acid in different concentrations ranging from 4% to 0.1%. Each solution is then dialysed against a PBS buffer having a pH of between 6.5 and 7. The degree of swelling is measured in order to control the final concentrations of each solution. Dialysis is stopped when the pH of the solutions is between 6.0 and 7.4. Each solution is sterilised by autoclave (121° C. for 15 minutes). The solutions are used as they are without any other form of dilution, which preserves their pH.

The solutions obtained can gel in the presence of the physiological medium or a culture medium.

### Example 2: Effect of Various Dilutions of Chitosan on the Proliferation, Differentiation and Capture of Fatty Acid by the 3T3L1 Murine Adipocyte Line

### Equipment and Methods

Various chitosan solutions (2%, 1%, 0.75% and 0.5%) are prepared by dilution of the solutions obtained in example 1.

For example, a 2% (w/w) chitosan preparation obtained in example 1 (DA: 2%, Mw: 450000 g·mol⁻¹, Bioxis Pharmaceuticals S.A.S.) is diluted in a saline solution (NaCl at 300 mOsmol·l⁻¹) containing 1% (w/w) of acetic acid in a well (in 24-well plates) and then distributed over half of the other wells, dried, rinsed with PBS and then with the culture medium before cell seeding (3T3L1 murine adipocyte line ATCC CL-173™). When the cells are at confluence, differentiation is induced by adding a differentiation medium. The cells are then treated with a small dose of oleic acid (OA, 2.5 μM) for 6 hours. The cells are put back in a culture medium without OA until the next day in order to avoid any background noise from the OA. Proliferation is analysed by phase contrast microscopy (×20): the images are taken at various times (2 days and 5 days post seeding), at an identical position for each well. The differentiation and the storage of oleic acid are analysed in fluorescence microscopy: the cells are fixed and then marked with AdipoRed (as identified in the figures) revealing the presence of fatty acids in the form of triglycerides (TGs) stored in a plurality of droplets during differentiation: fusing in a single droplet when the adipocytes are mature) and with Hoechst 3352 (red experimentally) for marking the nuclei. The marking is observed in fluorescence microscopy and quantification carried out by imaging. The imaging and the fluorescence quantification are carried out on a real-time imaging station. The controls are fixed before the induction of differentiation (confluence, DO).

### Results

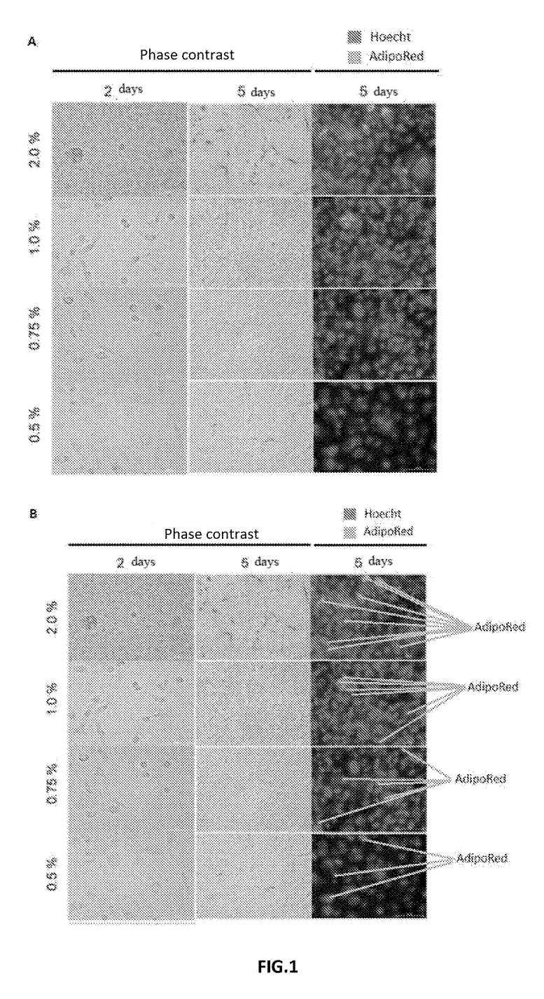

Two days after seeding, for all concentrations, the 3T3L1 cells are capable of proliferating and achieving confluence in 5 days (FIGS. 1A and 1B). Thus, at the concentrations tested, the adipocytes increase in number and volume. The morphology of the cells does however suggest an aggressive or even toxic medium for the 2% condition.

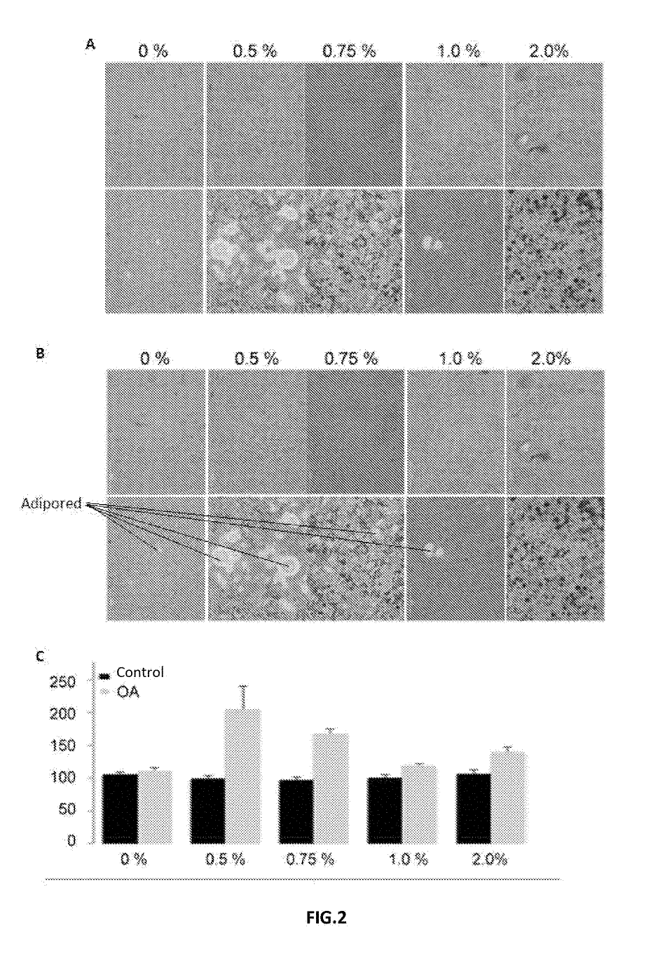

FIG. 2 shows the influence of the chitosan concentration on the storage of oleic acid for the 3T3L1 cells during adipocyte differentiation. Thus the same cells as those tested in FIG. 1 are used in the presence of oleic acid (fatty acid that the adipocyte cells have an ability to capture). The AdipoRed marking (FIGS. 2A and 2B) show an accumulation of fatty acids when the cells are in the presence of chitosan at 0.5%, 0.75% and 1% compared with the conditions not containing chitosan. Thus, in the presence of chitosan, the 3T3L1 cells capture more oleic acid than the reference. Quantification of this accumulation demonstrates that it is maximum when the cells are cultivated in the presence of chitosan at 0.5% (FIG. 2C).

### Conclusion

3T3L1 cells are capable of proliferating in the presence of chitosan. After two days in culture, the presence of chitosan does however appear to have a toxic effect on the 3T3L1 line for concentrations greater than or equal to 2%. Chitosan assists the capture of fatty acids in a dose-dependent fashion. Among the solutions tested, it appears that it is the 0.5% solutions that have the best efficacy. This capture would allow an increase in the volume of the adipocytes and therefore, by extrapolation, of the fatty tissues in vivo.

### Example 3: Effect of Various Dilutions of Chitosan on the Capture of Fatty Acid in the Presence of Fibronectin and Collagen by the 3T3L1 Murine Adipocyte Line

### Equipment and Methods

Various chitosan solutions (2%, 1% 0.75% and 0.5%) are prepared by diluting the solutions obtained in example 1.

For example, a 2% (w/w) chitosan preparation obtained in example 1. For this purpose, a 2% (w/w) chitosan preparation (DA: 2%, Mw: 450000 g·mol⁻¹, Bioxis Pharmaceuticals S.A.S.). The solutions were used as they are without undergoing any predilution in acid saline solution. In addition, a solution based on 2 mg/ml collagen I+1 mg/ml fibronectin I+2% chitosan (denoted Col FN Chi2) is also evaluated. The compositions are distributed in a 96-well plate after cell seeding (3T3L1 murine adipocyte line, ref ATCC CL-173™). When the cells are at confluence, differentiation is induced by adding a differentiation medium. The cells are then treated with a low dose of oleic acid (OA, 2.5 VM) for 6 hours. The cells are put back in a culture medium without oleic acid until the next day before observation on Cytation 3. The size distribution of the lipid droplets (the volume of the adipocytes) is observed.

### Results

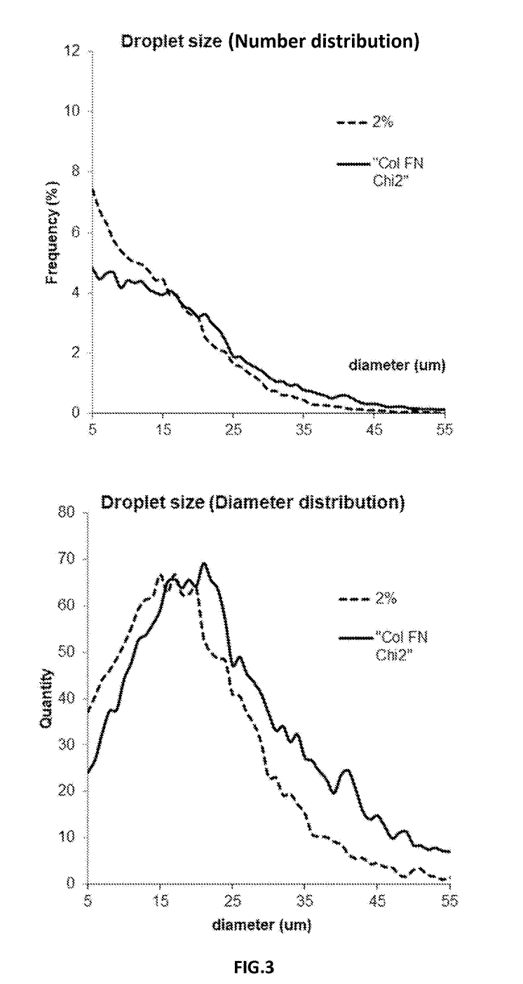

FIG. 3 shows that the 2 mg/ml collagen I+1 mg/ml fibronectin I+2% chitosan solution (denoted Col FN Chi2) has fewer adipocytes with a size below 15 μm and more adipocytes with a size of between 15 and 55 μm than the 2% chitosan solution. Adding compounds of the extracellular matrix such as collagen or fibronectin therefore promotes the increase in the number and average size (and therefore of the volume) of the lipid droplets.

### Conclusion

Chitosan alone increases the size of lipid droplets. This effect is further increased in the presence of fibronectin and collagen.

1. Aqueous chitosan composition comprising between 0.1% and 5% by weight chitosan, with respect to the total weight of the composition.

2. A method for treating or combating against skin ageing, for filling in skin defects such as wrinkles or for repairing and reconstructing cutaneous or subcutaneous tissues comprising administering to a subject in need thereof an effective amount of the aqueous chitosan composition of claim 1.

3. The method according to claim 2, wherein the aqueous composition comprises between 0.2% and 3% by weight, preferably between 0.5% and 2% by weight, more preferentially between 0.5% and 1.9% by weight chitosan, with respect to the total weight of the composition.

4. The method according to claim 2, wherein the aqueous composition has a pH of less than 8.5, advantageously between 4 and 7.6, more advantageously between 5.5 and 7.5, preferably between 6.2 and 6.8.

5. The method according to claim 2, wherein the chitosan has a degree of acetylation of less than 30%, advantageously less than 15%, and preferably less than 10%.

6. The method according to claim 2, wherein the aqueous composition further comprises at least one component of the extracellular matrix, and/or at least one substituent of the extracellular matrix.

7. The method according to claim 6, wherein the component of the extracellular matrix is chosen from proteins and the glycosylated derivatives thereof, in particular chosen from collagen, elastin, fibronectin, laminin or mixtures thereof.

8. The method according to claim 6, wherein the composition contains between 0.01% and 5% by weight, with respect to the total weight of the composition, of at least one component of the extracellular matrix and/or of at least one substituent of the extracellular matrix, advantageously between 0.1% and 4% by weight, more advantageously between 0.2% and 3% by weight, even more advantageously between 0.3% and 2% by weight, or even between 0.5% and 1% by weight.

9. The method according to claim 2, wherein the aqueous composition is liquid or in gel form.

10. The method according to claim 2, wherein the aqueous composition further comprises at least one active compound such as an analgesic compound, a local anaesthetic, such as lidocaine, mepivacaine, bupivacaine or ropivacaine, an angiogenic compound, or an active compound of the growth factor or bioactive oligosaccharide type.

11. The method according to claim 2, wherein the aqueous composition is formulated so as to be administered by intradermal injection, by subcutaneous or intraperitoneal injection.

12. A dermatological composition comprising the aqueous chitosan composition of claim 1.

13. A bioresorbable or partially resorbable implant comprising the aqueous chitosan composition of claim 1.

14. A method for reducing the toxicity with regard to adipocytes of an aqueous chitosan composition comprising between 0.1% and 5% by weight chitosan with respect to the total weight of the composition to said composition comprising adding at least one component of the extracellular matrix, and/or at least one substituent of the extracellular matrix.

15. A method for increasing the volume and/or number of adipocytes comprising administering to a subject in need thereof an effective amount of the aqueous chitosan composition of claim 1.

16. A method device comprising the aqueous chitosan composition of claim 1.
